(19) United States
(12) Patent Application Publication
(10) Pub. No.: US 2010/0304017 A1
(43) Pub. Date: Dec. 2, 2010

(54) URETHANE AND OLIGOURETHANE DERIVATIVES AND CORRESPONDING USES AND METHODS FOR PRODUCING WATER MARKS USING THE OFFSET PRINTING TECHNIQUE

(76) Inventors: Antonio Oliva Gurgui, Castelltercol (Barcelona) (ES); Josep Rocas Sorolla, Castelldefels (Barcelona) (ES); Jose Manuel Vilchez Martin, Esparraguera (Barcelona) (ES)

Correspondence Address: THE LAW OFFICES OF THOMAS L. ADAMS, 120 EAGLE ROCK AVENUE, Suite 130, EAST HANOVER, NJ, 07936, US

(21) Appl. No.: 12/599,125

(22) PCT Filed: May 6, 2008
(86) PCT No.: PCT/EP2008/003619

§ 371 Date: Feb. 5, 2010

(30) Foreign Application Priority Data
May 7, 2007 (ES) 200701203

(51) Int. Cl. C07C 271/10; C09D 5/00; B41M 5/00

(52) U.S. Cl. 427/145; 560/115; 106/31.97

(57) ABSTRACT

Urethane and oligourethane derivatives and corresponding uses and methods of producing watermarks using the offset printing technique. The invention relates to compounds having general formula (R—CO—NH—X—NH—CO—O—(-A-O—CO—NH—X′—NH—CO—O—)ₙ—R′(I) where, for example, R and R′ are C₃-C₁₈ alkyl radicals or the radical from the dimer diol when loosing an OH group, X and X′ are radicals from an aliphatic diisocyanate, A is a C₃-C₁₈ alkylen radical, n is 0 or 1. The molecular weight of the compound is less than 2000 and the NCO value is equivalent to 0. The invention also relates to compositions containing this compound, to methods for producing watermarks using this compound and to uses of these compounds to produce watermarks both in laminar cellulosic materials and in laminar textile materials.

### FIELD OF THE INVENTION

The invention relates to compounds and/or compositions for producing watermarks in laminar cellulosic materials, and in particular the invention relates to compounds having the general formula (I)

R—O—CO—NH—X—NH—CO—O—(-A-O—CO—NH—X′—NH—CO—O—)ₙ—R′ (I)

and compositions containing it. The invention also relates to methods for producing watermarks on laminar cellulosic materials and/or in laminar textile materials, particularly using the so-called offset system, and to uses of compounds having general formula (I) and compositions containing them for producing watermarks on laminar cellulosic materials and/or in laminar textile materials.

### STATE OF THE ART

Various compositions and methods for producing watermarks are known. However it is usually necessary to use compositions containing substances that are more or less toxic and/or difficult to handle. In addition, these methods are suitable for mass production series, and they are expensive to apply to small and medium production series.

The compositions described in Spanish Patent No. P200601897, in the name of the same applicant, are also known. These compositions are particularly effective when using the flexography printing technique.

One of the problems that must be overcome in order to obtain a good watermark is ensuring the chemical watermark penetrates well into the paper, in order to achieve transparency, while also ensuring it fixes well onto the paper or cellulosic material. Another problem to be solved is avoiding any sideways migrations. In the above-mentioned Spanish Patent No. P200601897, polymers or mixtures thereof are used which, in principle, fix the product that provides the transparency to obtain penetration and fixing. However, when polymers are used there is the risk that they may remain on the surface (which makes the watermark visible) and, also, that they may not fix the transparency sufficiently.

It is also complicated to appropriately fix the product that provides the transparency or watermark on the paper, without using cross-linking agents that are generally based on harmful or toxic monomers or reactive products. Moreover, the stability of these mixtures or compositions is limited.

Other known alternatives that use reactive oligomers-monomers (with relatively low viscosity) suffer from the drawback that they require energy (generally ultraviolet light or other electromagnetic waves) or other complex systems to cross link at the exact moment, after penetrating the paper, without any sideways migration. Moreover, these monomers or oligomers are usually harmful. Also they are not very manageable as it is difficult to achieve, at the exact moment, both penetration without sideways migration and fixation, because they usually react quickly and remain outside the paper or on the surface thereof.

### DISCLOSURE OF THE INVENTION

The purpose of the invention is to overcome these drawbacks. This aim is achieved by means of the compound having the general formula

R—O—CO—NH—X—NH—CO—O—(-A-O—CO—NH—X′—NH—CO—O—)ₙ—R′ (I)

where

R and R′ are C₃-C₁₈ alkyl radicals, C₃-C₁₈ hydroxyalkyl aliphatic radicals, the radical coming from the dimer diol when losing one OH group, or mixtures of the former,

X y X′ are radicals from an aliphatic diisocyanate with a molecular weight less than 1000, preferably less than 500,

A is a C₃-C₁₈ alkylen radical (from an alkyldiol), the radical from the dimer diol when losing two OH groups, or mixtures of the former,

n is 0 or 1,

where the molecular weight of the compound is less than 2000, preferably less than 1000,

and where the value of NCO is equivalent to 0.

In fact, the compounds having general formula (I) allow a combination of properties to be obtained, which makes them particularly useful in the production of watermarks. They are urethanes or oligourethanes which, in principle, are not reactive, as they do not have free NCO groups. Nevertheless, once applied, and due to their urethanic structure they fix well and are relatively solid. These compounds have a low viscosity, penetrate well in laminar cellulosic materials, present little sideways migration and are resistant to water and organic solvents (thanks to the affinity between the oligourethanes and the cellulosic material), they are hydrophobic and soluble in non-polar organic solvents, they are easy to handle, etc. Moreover, they have numerous —CH₂-groups, which means that they have a low refraction index, in particular, their refraction index is between 1.4 and 1.6. Preferably they have an index close to 1.54 (for example, between 1.52 and 1.56), and therefore a compromise has to be reached between the ability to fix to the cellulosic material, the refraction index and the viscosity. All this allows a series of advantages to be obtained, such as obtaining a highly transparent watermark, with well defined contours, which are affected very little if a drop of water or organic solvents falls on them (in other words, the watermark is very solid and fixes well to the cellulosic material) and afterwards it is easy to clean the machine and the tools used to produce the watermark. These compounds are also relatively simple and versatile.

The compound having general formula (I) will be more or less hydrophobic according to the radicals it contains. Generally, in this description and claims it must be understood that hydrophobic and insoluble in water are equivalent terms. Also, in this description and claims it is to be understood that a compound having general formula (I) is hydrophobic when it is hydrophobic enough to be able to be used in a conventional offset printing system. In this respect, a person skilled in the art is perfectly capable of determining whether a certain compound is suitable for use in an offset printing system and, therefore, is capable of determining whether a certain compound is hydrophobic according to this invention. In this respect, the aim of the invention is a compound having general formula (I), where n is 0 or 1, the molecular weight of the compound is less than 2000, preferably less than 1000, the NCO value is equivalent to 0, and R, R′, A, X and X′ are such that the compound is hydrophobic (is hydrophobic enough to be able to be used in an offset printing system).

As for the viscosity, it has been observed that this cannot be too low either, because then, during the printing, the rolls entrain too little material and therefore neither is the quality of the watermark satisfactory, because enough material (or, in more general terms, the laminar cellulosic material) must be provided so that it can be soaked sufficiently. In certain cases, this drawback can also be overcome by passing the paper through the offset machine several times.

The compound dimer diol is a diol containing 36 carbons that results from the dimerisation of an unsaturated fatty acid and the subsequent reduction of the two acid groups to alcohols to form the diol. It is also known as dimerol or dimer acid diol. It is assigned CAS No. (Chemical Abstracts Number) 147853-32-5. Commercially it is also known as Pripol 2033®, and is sold by Uniquema. In this description and claims, the indication that the dimer diol looses an OH group means that the urethane link is formed by an OH group from the dimer diol, whereby the dimer diol looses said OH group and remains linked to the oxygen by means of the corresponding carbon. Similarly, by saying that the dimer diol looses the two OH groups, it means that the two urethane links (the radical A has a urethane link at each end thereof) are formed by means of both OH groups, whereby the dimer diol looses both OH groups and remains linked to the respective oxygen molecules by means of the corresponding carbons.

Preferably the compound having general formula (I) only has additional nonpolar functional groups, preferably halogen or cyan radicals. In fact, in order to be applied in the offset printing technique it is essential that the compound is hydrophobic and, therefore, it is advantageous that it does not have polar functional groups (apart from the urethane groups). It is particularly advantageous for the compound having general formula (I) not to have any additional functional group.

A preferable alternative is that the compound (I) be linear. The linearity of the products produces two effects on the penetration capacity. When a product is linear it is more flexible and adapts better esterically to the substrate where it is applied, and if its functionality is correct, it penetrates more easily in comparison with a product that has a similar molecular weight and function, but is not linear. The linearity also influences viscosity, with equal molecular weight and functionality, generally, linear products are less viscose than cross-linked ones. Generally, the viscosity is a very important parameter when producing watermarks. If the viscosity is too low it may cause problems of insufficient solidity, excessive sideways migration, etc. Moreover, if the viscosity is too high it may cause problems of insufficient penetration. A linear compound, in general, will be less viscose than a branched compound. For its part, the branches will affect viscosity more or less depending on the constitution thereof: the presence of polar lateral groups (such as for example the —OH type) will consequently cause hydrogen bridge, etc. (forming a certain degree of interactions) type links to be formed, which largely affects viscosity. In this description and claims it must be understood that a linear compound is that which does not have any branches or cross-links, although the compound can include one or two cycles of 4, 5 6 or 7 carbons, such as for example in the particular cases cited below (dimer diol, isophorone, dicyclohexylmethyl, tetramethylxylilene, etc.). It is particularly advantageous that R and R′ be linear, specifically that they be strictly linear (without any type of cycle).

Advantageously the C₃-C₁₈ hydroxyalkyl aliphatic radical is a hydroxyalkyl radical from the group made up of 3-hydroxypropyl, 4-hydroxybutyl, 5-hydroxypentyl-, 6-hydroxyhexyl, 7-hydroxyheptyl, 8-hydroxyoctyl, 9-hydroxynonyl, 10-hydroxydecyl, 11-hydroxyundecyl, 12-hydroxydodecyl, 13-hydroxytridecyl, 14-hydroxytetradecyl, 15-hydroxypentadecyl, 16-hydroxyhexadecyl, 17-hydroxyheptadecyl, and 18-hydroxyoctadecyl. It is particularly advantageous that the C₃-C₁₈ hydroxyalkyl radical be a hydroxyalkyl radical from the group made up of 6-hydroxyhexyl, 8-hydroxyoctyl, 10-hydroxydecanyl, and 12-hydroxydodecyl, in other words, originating from the 1,6-hexanodiol, 1,8-octanodiol, 1,10-decanodiol, 1,12 dodecanodiol. Nevertheless, the possibility should not be ruled out that they come from diols with near OH, such as 1,2-hexanodiol, whose radical would be 2-hydroxy-1-hexyl for example and so on successively. In this respect, it is also advantageous that the C₃-C₁₈ hydroxyalkyl aliphatic radical be a hydroxyalkyl radical from a diol from the group made up of 1,2-dodecanodiol, 1,2-decanodiol, 1,2-octanodiol, and 1,2-hexanodiol. Nevertheless this does not rule out the possibility that the OH group is in any other position. Alternatively, it is particularly advantageous that the C₃-C₁₈ alkyl radical be an alkyl radical from the group made up of hexyl, octyl, decyl, and dodecyl. In fact, these radicals make it possible to obtain an optimum combination of the properties mentioned above: viscosity, penetration capacity, solidity, sideways migration, refraction index, handling capacity, cost, etc..

Preferably R and R′ are the radical from the dimer diol when loosing an OH group, in other words, when it reacts through one of its OH groups, leaving the other one free.

Preferably X and X′ are hexamethylene, isophorone, dicyclohexylmethyl, tetramethylxylilene, xylilene, or trimethylhexamethylene, in other words, the radicals that are obtained when reacting the isocyanates, hexamethylene diisocyanate, isophorone diisocyanate (IPDI), dicyclohexylmethyl diisocyanate, tetramethylxylilene diisocyanate, xylilene diisocyanate, or trimethylhexamethylene diisocyanate with alcohols.

Another preferable alternative of compound (I) is when n is 0.

Generally, the inventors have observed that the basic requirement for the compound having general formula (I) to be suitable for producing watermarks using offset is that it has a certain number of carbons (preferably —CH₂— groups). It must have at least 10 carbons, and preferably it must have at least 12 carbons (or —CH₂— groups). The distribution of these carbons between R, R′, A, X and X′ is less important. This minimum amount of carbons (or —CH₂— groups) is what makes it possible to obtain the properties of hydrophobicity, viscosity, penetrability, solidity, sideways migration and refraction index, which make the compounds suitable for making watermarks using offset. In other words, an alternative and similar way of presenting the invention is to say that its aim is a compound having general formula

R—O—CO—NH—X—NH—CO—O—(-A-O—CO—NH—X′—NH—NH—CO—O—)ₙ—R′ (I)

where

R and R′ are alkyl radicals, aliphatic hydroxyalkyl radicals, the radical coming from the dimer diol when loosing an OH group, or mixtures of the former,

X y X′ are radicals from an aliphatic diisocyanate with a molecular weight less than 1000, preferably less than 500,

A is a C₃-C₁₈ alkylen radical (from an alkyldiol), the radical from the dimer diol when losing two OH groups, or mixtures of the former,

n is 0 or 1,

where said compound has at least 10 carbon atoms, preferably at least 12 carbon atoms,

where the molecular weight of said compound is less than 2000, preferably less than 1000,

and where the NCO value is equivalent to 0. Advantageously it has at least 10 —CH₂— groups and preferably it has at least 12 —CH₂— groups. In this respect, it is important to highlight that the compound overall must be hydrophobic, but the distribution of the carbons (or the —CH₂— groups) between R, R′ and A can be done in several ways. Preferably, but not necessarily, most of the carbons are at the ends of the compound, so the polarity of said compound remains concentrated in its central portion.

The compounds according to the invention also have the advantage that they are very solid with respect to light and chemical products. In fact, it has been observed that, as they do not have double links, they do not have oxidation phenomena (for example, with atmospheric air) and do not develop colours that make the watermark visible. The compounds according to the invention also have a suitable reactivity, whereby they are fluid enough to penetrate well, but not so fluid as to noticeably migrate sideways before fixing.

Advantageously compound (I) contains a colouring agent. In fact, a preferable embodiment of the invention is obtained when a colouring agent is included, so that the resulting watermark is coloured. The colouring agent can be included such that it is linked chemically to the compound (so that the compound, overall, comprises the colouring agent), or it can be included in the composition used to produce the watermark, like one more component thereof. Examples of these colouring agents are the Savinyl® colouring agents that are sold by the Pigments and Additives division of the Clariant Group. In the event that the colouring agent is linked chemically to compound (I), it is advantageous that it be linked to the compound through an urethane link formed by an —OH group of the colouring agent and an NCO group of the compound. Examples of the colouring agents of this type are the Reactint® colouring agents, sold by Milliken Chemical, which is a division of Milliken & Company. Nevertheless, the chemical link increases the molecular weight and consequently, in certain cases, hinders penetration. It may also be interesting to combine both types of colouring agents, according to the desired colour to be obtained.

Preferably the compound contains the colouring agent in a proportion that is less than or equivalent to 0.5% by weight, preferably less than or equivalent to 0.4% by weight with respect to the total weight of the compound, both if the colouring agent is linked chemically to the compound and if both are dissolved and/or dispersed in a composition. It has been observed that when higher percentages are used, the quality of the watermark transparency is reduced, because the refraction index of the colouring agent (particularly red and blue) is greater that the refraction index of the cellulosic material, and so high concentrations of these colouring agents reduce the transparency obtained with the compound that does not contain any colouring agent.

A preferable embodiment of the invention is obtained when a composition is prepared and used, which contains at least a first compound having general formula (I) according to the invention, where R, R′, X, X′, A and n have the general meaning indicated above, and a nonpolar solvent, preferably from the group made up of benzene mono- or polysubstituted with C₁-C₄ alkyl radicals, turpentine, and mixtures of the above, where the benzene mono- or polysubstituted with C₁-C₄ alkyl radicals is preferably toluene, xylene or ethylbenzene. In fact, to produce and use compound (I) appropriately it is advisable to dilute it in a solvent. On the one hand, this facilitates the production thereof and, on the other hand, it facilitates its application and penetration in the cellulosic material.

An example of a particularly interesting mixture of solvents is the solvent known commercially as hydrowash®, which is a mixture of alkylbenzenes.

Preferably the composition comprises at least one second compound having general formula (I), where the second compound has a different molecular weight to the first compound. This way it is possible to obtain a combination of properties that would be difficult to obtain with one single compound, such as for example, obtaining a balance between low viscosity and good fixation. The mixture is normally made between a product that adheres or fixes well to the cellulose (but which is rather viscose for some types of paper, particularly thick and dense paper) and another that is more fluid and which even provides more transparency. Advantageously in the first compound R and R′ are the radical from the dimer diol when loosing an OH group, X is isophorone, n is 0, and in the second compound R and R′ are octyl, X is isophorone and n is 0, where the proportion between the first compound and the second compound is between 40:60 by weight and 60:40 by weight, and it has between 20% and 35% by weight of solvent, preferably between 25% and 30% by weight of the solvent.

Preferably, in the composition, the solvent content is less than or equivalent to 30% by weight with respect to the total weight of the compound having general formula (I), and very preferably it is less than or equivalent to 20% by weight with respect to the total weight of the compound having general formula (I). These concentrations are the most suitable to use when producing watermarks. If the solvent content is greater the watermark is not transparent enough, because the pores of the cellulosic material are not filled sufficiently with the compound having general formula (I). Moreover, if the viscosity of the composition is too low, the offset rollers pick up a smaller amount of the composition, which is not enough to form a good watermark.

Advantageously, the composition comprises a colouring agent, and preferably contains it in a proportion less than or equivalent to 0.3% by weight, preferably less than or equivalent to 0.15% by weight with respect to the total weight of the compound having general formula (I). Specifically, it is particularly advantageous that the composition has between 0.15% and 0.05% by weight of the yellow colouring agent Savinyl Yellow RLS® (particularly 0.1% by weight), or between 0.07% and 0.03% by weight of the blue colouring agent Savinyl Blue GLS p® (particularly 0.05% by weight), or between 0.05% and 0.02% by weight of the red colouring agent Savinyl Red 3BLS p® (particularly 0.035% by weight).

As can be seen, the compounds according to the invention have the advantage that their degree of viscosity and reactivity are suitable for producing watermarks, because they are easier to handle, penetrate suitably and do not remain on the surface. They do not require much solvent to reduce their viscosity, which means that the intensity of the transparency, when the solvent has evaporated, is high. Using a high concentration product, with high penetration (to create a good transparency) which, in turn, has enough affinity, once the solvent has evaporated, to fix satisfactorily to the cellulosic material and does not show any sideways migration, obtains a high transparency watermark with great clarity.

The object of the invention is also a method for producing a watermark on a laminar cellulosic material characterized in that it comprises a stage of applying a compound having general formula (I), where R, R′, X, X′, A and n have the general meaning indicated above, onto the laminar cellulosic material using the offset system.

Moreover the object of the invention is a method for producing a watermark on a laminar cellulosic material, characterized in that it comprises a stage of applying a composition according to the invention onto the laminar cellulosic material using the offset system.

The object of the invention is also the use of a compound having general formula (I), where R, R′, X, X′, A and n have the general meaning indicated at the beginning, for producing a watermark on a laminar cellulosic material.

The object of the invention is also the use of a composition according to the invention for producing a watermark on a laminar cellulosic material.

Conventionally, the watermark is produced during the paper production process. To do this, rollers are used bearing relief drawings, which are generally arranged at a point before the continuous roller of wet paper enters the driers. This way, the cellulose fibres of the continuous roller move horizontally (in the direction of the actual paper) so that the paper is made thinner and, consequently, more transparent in the area of the filigree or watermark. Therefore, the rollers with relief drawings “deform” the wet paper so that this deformation remains permanently on the paper after drying. Another conventional method for producing filigrees or watermarks, and which is also performed while producing the paper, consists in using a web adjusted around a roller, which bears a relief drawing or shape of the watermark. This relief drawing comes into contact with the wet paper, which still has a certain degree of plasticity, which allows it to be compressed. The area of compressed paper has a smaller amount of trapped air and, therefore, less light refraction, thereby increasing its transparency. Consequently, watermarks are currently produced during the paper production process and they are in white and black (in other words, generally, in a single colour according to the colour of the paper) because they only play with the transparency, shade and opaqueness.

This invention enables watermarks to be produced on papers (generally, laminar cellulosic materials) that have already been produced, in a subsequent stage corresponding to the printing. Moreover, this invention allows coloured watermarks to be produced. In fact, the compounds and compositions according to the invention are suitable for use as inks for the offset system. This printing technique uses liquid inks (with a low viscosity) that essentially consist of a solvent and a series of additives (colouring agents, resins, pigments, waxes or plastifying agents, etc.). The liquid inks are applied to the cellulosic support material and the solvent evaporates. By using the compounds and compositions according to the invention, these penetrate inside the cellulosic material reducing their refraction and thereby producing a watermark.

Finally, it must be taken into consideration that, although it is advantageous to use the compounds to produce watermarks with the offset system, this is not the only application possible. In fact, by appropriately selecting the radicals R, R′, A, X and X′ the compound having general formula (I) can be suitable for use in the production of watermarks using flexography, in a way equivalent to that described in said Spanish patent application P200601897. Also, by choosing polar solvents, compositions could be obtained that contain the compound having general formula (I) and which are suitable for producing watermarks using flexography.

The object of the invention is also a method for producing a watermark on a laminar textile material characterized in that it comprises a stage of applying a compound having general formula (I) according to the invention, where R, R′, X, X′, A and n have the general meaning indicated above, or a composition according to the invention, on the laminar textile material using the offset system.

Also the object of the invention is the use of a compound having general formula (I) according to the invention, where R, R′, X, X′, A and n have the general meaning indicated above, or a composition according to the invention, for producing a watermark on a laminar textile material.

In fact, it has been observed that, surprisingly, it is possible to produce watermarks on laminar textile materials in a way similar to how they are produced on laminar cellulosic materials. Generally, the laminar textile material can be of any kind.

A preferable solution is that the laminar textile material be woven (understood to be conventional weaving, in other words, with warp and weft), although another advantageous solution is that the laminar textile material be a non-woven fabric. Generally, any laminar textile material composed of fibres is valid, where the inter-fibre spaces are no larger than the size of the fibre diameter (or than the diameter of the largest fibres, if there is a mixture of different diameter fibres).

The fibres can be any artificial or natural fibre, such as polyester, polyamide (nylon), polyurethanes (lycra), acrylic fibre, regenerated cellulose fibres (rayon), natural fibres of vegetable origin (cotton, linen, etc.), or of animal origin (wool, silk, etc.). Although the fabric can contain mixtures of different types of fibres, it is advantageous that it be of one single type of fibre or, at least, that the different types of fibre have a similar refraction index. Preferably the refraction index of any of the fibres making up the laminar textile material must be between 1.35 and 1.65. At any event, it is advantageous that the fibres do not have an opaque material filler (such as, for example, calcium carbonate or calcium sulphate) and/or metallic oxides (such as, for example, titanium oxide) and/or other compounds with a high refraction index (higher than 1.65).

The watermark can be used as a quality control, which makes it possible to certify the origin of production, like a “first use” control (if using watermarks that have precisely a low fixation ability so that they are removed or noticeably deteriorated when washed), etc. In these cases, preferably the watermark will be arranged on the label, and so it is preferable that the laminar textile material be polyester.

In other cases it may be interesting for the watermark to be part of the decoration on a clothing garment. In these cases it will be particularly interesting for the laminar textile material to be cotton, which is a standard material for making T-shirts and the like.

### DETAILED DESCRIPTION OF SOME EMBODIMENTS OF THE INVENTION

### Example 1

### IPDI Plus Caproylic Alcohol with 10% Excess of Caproylic Alcohol

178.97 g of caproylic alcohol (1-hexanol, 1.7532 eq OH) and 0.17 g of DBTL (dibutyl tin dilaurate) (0.048% total weight) were loaded into a four-mouth reactor fitted with a nitrogen inlet and a thermometer and shaker. The thermostat was set at 90° C. with a shaking speed of 90-100 r.p.m in an inert nitrogen atmosphere. Once the temperature was reached, 177.3 g of IPDI (1.5952 eq NCO) were added, drop by drop, making sure that the temperature did not exceed 100° C. due to the exothermic conditions. Once the IPDI addition was complete, it was left to react at 90° C. for 60 minutes and it was checked that the product's NCO was zero by using the standard dibutylamine method (as defined in the UNE-EN 1242 standard). When the NCO was zero, 39.59 g of toluene were added to the product to take it to 90% in solids.

This compound is a preferred embodiment of the invention, where R and R′ are hexyl, X is isophorone and n is 0.

### Example 2

### IPDI plus Caprylic Alcohol with 2% Excess of Caprylic Alcohol

110.07 g of caprylic alcohol (1 n-octanol, 0.8451 eq OH) and 0.097 g of DBTL (0.048% total weight) were loaded into a four-mouth reactor fitted with a nitrogen inlet and a thermometer and shaker). The thermostat was set at 90° C. with a shaking speed of 90-100 r.p.m in an inert nitrogen atmosphere. Once the temperature was reached, 92.09 g of IPDI (0.8286 eq NCO) were added, drop by drop, making sure that the temperature did not exceed 100° C. due to the exothermic conditions. Once the IPDI addition was complete, it was left to react at 90° C. for 60 minutes and it was checked that the product's NCO was zero by using the standard dibutylamine method (UNE-EN 1242). When the NCO was zero, 22.46 g of toluene were added to the product to take it to 90% in solids.

### Example 3

### IPDI Plus Caprylic Alcohol with 10% Excess of Caprylic Alcohol

118.07 g of caprylic alcohol (1 n-octanol, 0.9114 eq OH) and 0.1 g of DBTL (0.048% total weight) were loaded into a four-mouth reactor fitted with a nitrogen inlet and a thermometer and shaker). The thermostat was set at 90° C. with a shaking speed of 90-100 r.p.m in an inert nitrogen atmosphere. Once the temperature was reached, 92.09 g of IPDI (0.8286 eq NCO) were added, drop by drop, making sure that the temperature did not exceed 100° C. due to the exothermic conditions. Once the IPDI addition was complete, it was left to react at 90° C. for 60 minutes and it was checked that the product's NCO was zero by using the standard dibutylamine method (UNE-EN 1242). When the NCO was zero, 23.42 g of toluene were added to the product to take it to 90% in solids.

### Example 4

### IPDI Plus Caprylic Alcohol with 20% Excess of Caprylic Alcohol

129.5 g of caprylic alcohol (1 n-octanol, 0.9943 eq OH) and 0.11 g of DBTL (0.048% total weight) were loaded into a four-mouth reactor fitted with a nitrogen inlet and a thermometer and shaker. The thermostat was set at 90° C. with a shaking speed of 90-100 r.p.m in an inert nitrogen atmosphere. Once the temperature was reached, 92.09 g of IPDI (0.8286 eq NCO) were added, drop by drop, making sure that the temperature did not exceed 100° C. due to the exothermic conditions. Once the IPDI addition was complete, it was left to react at 90° C. for 60 minutes and it was checked that the product's NCO was zero by using the standard dibutylamine method (UNE-EN 1242). When the NCO was zero, 24.62 g of toluene were added to the product to take it to 90% in solids.

The compound of Examples 2, 3 and 4 is a preferable embodiment of the invention, where R and R′ are octyl, X is isophorone and n is 0.

Moreover, this compound is used to prepare a preferred composition which, in addition to this compound, has as solvent between 4% and 8% by weight of toluene and between 7% and 11% by weight of a mixture of benzenes mono- or polysubstituted with C₁-C₄ alkyl radicals, preferably 6% by weight of toluene and 9% by weight of a mixture of benzenes mono- or polysubstituted with C₁-C₄ alkyl radicals.

Another preferred composition is the one which, in addition to the compound in Examples 2, 3 and 4, and in addition to between 8% and 12% by weight of toluene and between 3% and 7% by weight of a mixture of benzenes mono- or polysubstituded with C₁-C₄ alkyl radicals as solvent (preferably 10% by weight of toluene and 5% by weight of a mixture of benzenes mono- or polysubstituted with C₁-C₄ alkyl radicals), also contains between 8% and 12% by weight of octanol. In fact, the inventors have observed that, in certain cases, it is advantageous to put a certain excess of the product that generates the R and/or R′ radical. This excess has several advantages: on the one hand it increases the speed of the reaction, on the other hand, it ensures that the reaction is complete (in other words, that no non-reacted isocyanates remain) and, on the other hand, the excess product helps with the solvent to obtain a composition that has the desired properties. Advantageously, the composition has between 2% and 20% by weight in excess of the compound that produces the R and/or R′ radical. In particular, in Examples 2, 3 and 4, the R radical is octyl, and the product that generates it is 1 n-octanol. In this particular case, it is also possible to reduce the amount of the solvent, so that it has between 0% and 5% by weight of toluene, and increase its content by 1 n-octanol, so that the composition has between 15% and 25% by weight of 1 n-octanol. Similarly, an advantageous composition can be obtained from the compound in Example 1, (R and R′ are hexyl, X is isophorone, n is 0) which, in addition to the solvent, has a content of between 8% and 12% by weight of 1-hexanol

### Example 5

### IPDI Plus Nafol 10D by Sasol with 2% Excess of Nafol 10D

239,69 g of Nafol 10D sold by the company Sasol (decyl alcohol 94.6%, 1.5143 eq OH) and 0.19 g of DBTL (0.048% total weight) were loaded into a four-mouth reactor fitted with a nitrogen inlet and a thermometer and shaker. The thermostat was set at 90° C. with a shaking speed of 90-100 r.p.m in an inert nitrogen atmosphere. Once the temperature was reached, 165.01 g of IPDI (1.4846 eq NCO) were added, drop by drop, making sure that the temperature did not exceed 100° C. due to the exothermic conditions. Once the IPDI addition was complete, it was left to react at 90° C. for 60 minutes and it was checked that the product's NCO was zero by using the standard dibutylamine method (UNE-EN 1242). When the NCO was zero, 44.97 g of toluene were added to the product to take it to 90% in solids.

### Example 6

### IPDI Plus Nacol 12-96 by Sasol with 2% Excess of Nacol 12-96

255.39 g of Nacol 12-96 by Sasol (laurylic acid 98.5%, 1.3731 eq OH) and 0.19 g of DBTL (0.048% total weight) were loaded into a four-mouth reactor fitted with a nitrogen inlet and a thermometer and shaker. The thermostat was set at 90° C. with a shaking speed of 90-100 r.p.m in an inert nitrogen atmosphere. Once the temperature was reached, 149.62 g of IPDI (1.3461 eq NCO) were added, drop by drop, making sure that the temperature did not exceed 100° C. due to the exothermic conditions. Once the IPDI addition was complete, it was left to react at 90° C. for 60 minutes and it was checked that the product's NCO was zero by using the standard dibutylamine method (UNE-EN 1242). When the NCO was zero, 45.00 g of toluene were added to the product to take it to 90% in solids.

### Example 7

### IPDI Plus Isofol 12 by Sasol with 2% Excess of Isofol 12

255.39 g of Isofol 12 by Sasol (2-Butyl-1-octanol 98.7%, 1.3731 eq OH) and 0.19 g of DBTL (0.048% total weight) were loaded into a four-mouth reactor fitted with a nitrogen inlet and a thermometer and shaker. The thermostat was set at 90° C. with a shaking speed of 90-100 r.p.m in an inert nitrogen atmosphere. Once the temperature was reached, 149.62 g of IPDI (1.3461 eq NCO) were added, drop by drop, making sure that the temperature did not exceed 100° C. due to the exothermic conditions. Once the IPDI addition was complete, it was left to react at 90° C. for 60 minutes and it was checked that the product's NCO was zero by using the standard dibutylamine method (UNE-EN 1242). When the NCO was zero, 45.00 g of toluene were added to the product to take it to 90% in solids.

### Example 8

### IPDI Plus Cetylic Alcohol with 5% Excess of Cetylic Alcohol

200 g of cetylic acid (C₁₆ alcohol, 0.8249 eq OH) were loaded into a four-mouth reactor fitted with a nitrogen inlet and a thermometer and shaker. The thermostat was set at 80° C. with a shaking speed of 90-100 r.p.m in an inert nitrogen atmosphere. Once the temperature was reached, 45.84 g of IPDI (0.4125 eq NCO) were added, drop by drop, making sure that the temperature did not exceed 100° C. due to the exothermic conditions. Once the IPDI addition was complete, it was left to react at 80° C. for 60 minutes and it was checked that the product's NCO was zero by using the standard dibutylamine method (UNE-EN 1242). When the NCO was zero, 27.32 g of toluene were added to the product to take it to 90% in solids.

### Example 9

### IPDI Plus Mixture of Caprylic Alcohol and Caproylic Alcohol with 10% Excess of the Mixture of Alcohols

94.56 g of caprylic alcohol (1 n-octanol, 0.7260 eq OH), 74.05 g of caprylic alcohol (1-hexanol, 0.7254 eq OH) and 0.15 g of DBTL (0.048% total weight) were loaded into a four-mouth reactor fitted with a nitrogen inlet and a thermometer and shaker. The thermostat was set at 90° C. with a shaking speed of 90-100 r.p.m in an inert nitrogen atmosphere. Once the temperature was reached, 144.72 g of IPDI (1.3200 eq NCO) were added, drop by drop, making sure that the temperature did not exceed 100° C. due to the exothermic conditions. Once the IPDI addition was complete, it was left to react at 90° C. for 60 minutes and it was checked that the product's NCO was zero by using the standard dibutylamine method (UNE-EN 1242). When the NCO was zero, 35.04 g of toluene were added to the product to take it to 90% in solids.

### Example 10

### IPDI plus Nafol 810D by Sasol with 2% excess of Nafol 810D.

231.12 g of Nafol 810D by Sasol (1 n-octanol 46% and 1-decanol 53.6%, 1.5945 eq OH) and 0.19 g of DBTL (0.048% total weight) were loaded into a four-mouth reactor fitted with a nitrogen inlet and a thermometer and shaker. The thermostat was set at 90° C. with a shaking speed of 90-100 r.p.m in an inert nitrogen atmosphere. Once the temperature was reached, 173.74 g of IPDI (1.5632 eq NCO) were added, drop by drop; making sure that the temperature did not exceed 100° C. due to the exothermic conditions. Once the IPDI addition was complete, it was left to react at 90° C. for 60 minutes and it was checked that the product's NCO was zero by using the standard dibutylamine method (UNE-EN 1242). When the NCO was zero, 44.98 g of toluene were added to the product to take it to 90% in solids.

### Example 11

IPDI plus Nafol 1214S by Sasol with 2% excess of Nafol 1214S

258.65 g of Nafol 1214S by Sasol (laurylic alcohol 70.4%, and myristylic alcohol 28.6%, 1.3437 eq OH) and 0.19 g of DBTL (0.048% total weight) were loaded into a four-mouth reactor fitted with a nitrogen inlet and a thermometer and shaker. The thermostat was set at 90° C. with a shaking speed of 90-100 r.p.m in an inert nitrogen atmosphere. Once the temperature was reached, 146.42 g of IPDI (1.3174 eq NCO) were added, drop by drop, making sure that the temperature did not exceed 100° C. due to the exothermic conditions. Once the IPDI addition was complete, it was left to react at 90° C. for 60 minutes and it was checked that the product's NCO was zero by using the standard dibutylamine method (UNE-EN 1242). When the NCO was zero, 45.00 g of toluene were added to the product to take it to 90% in solids.

### Example 12

### IPDI Plus Mixture of Caprylic Alcohol and Cetylic Alcohol with 2% Excess of the Mixture of Alcohols

107.58 g of caprylic alcohol (1 n-octanol, 0.8260 eq OH), 200.22 g of cetylic alcohol (1-hexadecanol, 0.8259 eq OH) and 0.23 g of DBTL (0.048% total weight) were loaded into a four-mouth reactor fitted with a nitrogen inlet and a thermometer and shaker. The thermostat was set at 90° C. with a shaking speed of 90-100 r.p.m in an inert nitrogen atmosphere. Once the temperature was reached, 180 g of IPDI (1.6195 eq NCO) were added, drop by drop, making sure that the temperature did not exceed 100° C. due to the exothermic conditions. Once the IPDI addition was complete, it was left to react at 90° C. for 60 minutes and it was checked that the product's NCO was zero by using the standard dibutylamine method (UNE-EN 1242). When the NCO was zero, 54.2 g of toluene were added to the product to take it to 90% in solids.

### Example 13

### IPDI Plus Pripol 2033 by Degussa with 2% Excess of Pripol 2033.

200 g of Pripol 2033 sold by the company Degusta (c₃₆ fatty acid dimer or dimer diol, 0.7306 eq OH) were loaded into a four-mouth reactor fitted with a nitrogen inlet and a thermometer and shaker. The thermostat was set at 80° C. with a shaking speed of 90-100 r.p.m in an inert nitrogen atmosphere. Once the temperature was reached, 40.60 g of IPDI (0.3653 eq NCO) were added, drop by drop, making sure that the temperature did not exceed 100° C. due to the exothermic conditions. Once the IPDI addition was complete, it was left to react at 80° C. for 60 minutes and it was checked that the product's NCO was zero by using the standard dibutylamine method (UNE-EN 1242). When the NCO was zero, 26.73 g of toluene were added to the product to take it to 90% in solids.

This compound is a preferred embodiment of the invention, where R and R′ are the radical from the dimer diol when it looses an OH group, X is isophorone and n is 0. This compound is also the basis for a particularly advantageous composition which, in addition to the compound, has as solvent between 11% and 15% by weight of toluene and between 5% and 9% by weight of turpentine, preferably 13% by weight of toluene and a 7% by weight of turpentine.

### Example 14

### IPDI Plus Mixture of Caprylic Alcohol and Pripol 2033 by Degussa with 2% Excess of the Mixture

35.86 g of caprylic alcohol (1 n-octanol, 0.2753 eq OH) and 150.74 g of Pripol 2033 by Degussa (C₃₆ fatty acid dimer or dimer diol, 0.2753 eq OH) were loaded into a four-mouth reactor fitted with a nitrogen inlet and a thermometer and shaker. The thermostat was set at 90° C. with a shaking speed of 90-100 r.p.m in an inert nitrogen atmosphere. Once the temperature was reached, 60.0 g of IPDI (0.5398 eq NCO) were added, drop by drop, making sure that the temperature did not exceed 100° C. due to the exothermic conditions. Once the IPDI addition was complete, it was left to react at 90° C. for 60 minutes and it was checked that the product's NCO was zero by using the standard dibutylamine method (UNE-EN 1242). When the NCO was zero, 27.40 g of toluene were added to the product to take it to 90% in solids.

### Example 15

### IPDI Plus Mixture of Cetylic Alcohol and Pripol 2033 by Degussa with 2% Excess of the Mixture

78.99 g of cetylic alcohol (C₁₆ alcohol, 0.3258 eq OH) and 89.19 g of Pripol 2033 by Degusta (C₃₆ fatty acid dimer or dimer diol, 0.3258 eq OH) were loaded into a four-mouth reactor fitted with a nitrogen inlet and a thermometer and shaker. The thermostat was set at 90° C. with a shaking speed of 90-100 r.p.m in an inert nitrogen atmosphere. Once the temperature was reached, 35.5 g of IPDI (0.3194 eq NCO) were added, drop by drop, making sure that the temperature did not exceed 100° C. due to the exothermic conditions. Once the IPDI addition was complete, it was left to react at 90° C. for 60 minutes and it was checked that the product's NCO was zero by using the standard dibutylamine method (UNE-EN 1242). When the NCO was zero, 22.63 g of toluene were added to the product to take it to 90% in solids.

This compound is a preferred embodiment of the invention, where R is the radical from the dimer diol when it looses an OH group, R′ is hexadecyl, X is isophorone and n is 0.

### Example 16

### Molecule 2:1 of IPDI with 1,4-butanodiol and Finished with Caprylic Alcohol (2% excess).

28.94 g of 1,4-butanodiol (0.6423 eq PH) and 142.77 g of IPDI (1.2845 eq OH) were loaded into a four-mouth reactor fitted with a nitrogen inlet and a thermometer and shaker. The thermostat was set at 60° C. with a shaking speed of 90-100 r.p.m in an inert nitrogen atmosphere. Once the theoretical NCO was reached, measured by the standard dibutylamine method (UNE-EN 1242), 66.82 g of caprylic alcohol (1-octanol, 0.6551 eq OH) were added, and it was left to react at 80° C. until the product's NCO was zero, measured using the standard dibutylamine method (UNE-EN 1242). When the NCO was zero, 26.50 g of toluene were added to the product to take it to 90% in solids. In this case, A was butenyl, and R and R′ were octyl, in other words, A was less than R and R′.

### Example 17

### Molecule 2:1 of IPDI with Pripol 2033 by Degussa and Finished with Caproylic Alcohol (2% Excess).

177.12 g of Pripol 2033 by Degusta (C36 dimer of fatty alcohol or dimer diol, 0.6423 eq OH) and 142.77 g of IPDI (1.2845 eq NCO) were loaded into a four-mouth reactor fitted with a nitrogen inlet and a thermometer and shaker. The thermostat was set at 60° C. with a shaking speed of 90-100 r.p.m in an inert nitrogen atmosphere. Once the theoretical NCO was reached, measured by the standard dibutylamine method (UNE-EN 1242), 66.82 g of caproylic alcohol (1-hexanol, 0.6551 eq OH) were added, and it was left to react at 80° C. until the product's NCO was zero, measured using the standard dibutylamine method (UNE-EN 1242). When the NCO was zero, 42.97 g of toluene were added to the product to take it to 90% in solids. In this case

A was the radical from the dimer diol when it looses the two OH groups and R and R′ were hexyl, in other words, R and R′ were smaller than A.

1- Compound Having General Formula
R—O—CO—NH—X—NH—CO—O—(-A-O—CO—NH—X′—NH—CO—O—)ₙ—R′ (I)
where
R and R′ are C₃-C₁₈ alkyl radicals, C₃-C₁₈ hydroxyalkyl aliphatic radicals, the radical coming from the dimer did when losing one OH group, or mixtures of the former,
X y X′ are radicals from an aliphatic diisocyanate with a molecular weight less than 1000, preferably less than 500,
A is a C₃-C₁₈ alkylen radical (from an alkyldiol), the radical from the dimer diol when losing two OH groups, or mixtures of the former,
n is 0 or 1,
where the molecular weight of said compound is less than 2000, preferably less than 1000,
and where the value of NCO is equivalent to 0,

2- Compound according to claim 1, characterized in that R and R′ are the radical from the dimer diol when it looses an OH group and n is 0.

3- Compound according to claim 1, characterized in that R and R′ are the radical from the dimer diol when it looses an OH group, X is isophorone and n is 0.

4- Compound according to claim 1, characterized in that R is the radical from the dimer diol when it looses an OH group, R′ is hexadecyl, X is isophorone and n is 0.

5- Compound according to claim 1, characterized in that R and R′ are octyl, X is isophorone and n is 0.

6- Compound according to claim 1, characterized in that n is 1.

7- Compound according to claim 6, characterized in that it only has non-polar additional functional groups, preferably halogen or cyan radicals.

8- Compound according to claim 6, characterized in that it does not have any additional functional group.

9- Compound according to claim 6, characterized in that R and R′ are linear.

10- Compound according to any of the claims 6 to 9, characterized in that said C₃-C₁₈ hydroalkyl aliphatic radical is a hydroxyalkyl radical from the group made up of 3-hydroxypropyl, 4-hydroxybutyl, 5-hydroxypentyl-, 6-hydroxyhexyl, 7-hydroxyheptyl, 8-hydroxyoctyl, 9-hydroxynonyl, 10-hydroxydecyl, 11-hydroxyundecyl, 12-hydroxydodecyl, 13- hydroxytridecyl, 14-hydroxytetradecyl, 15-hydroxypentadecyl, 16-hydroxyhexadecyl, 17-hydroxyheptadecyl, and 18-hydroxyoctadecyl, preferably from the group made up of 6-hydroxyhexyl, 8-hydroxyoctyl, 10-hydroxydecanyl, and 12-hydroxydodecyl.

11- Compound according to any of the claims 6 to 9, characterized in that R and R′ are the radical from the dimer diol when it looses an OH group.

12- Compound according to any of the claims 6 to 11, characterized in that X and X′ are hexamethylene, isophorone, dicyclohexylmethyl, tetramethylxylilene, xylilene, or trimethylhexamethylene.

13- Compound according to any of the claims 6 to 9 and 12, characterized in that said C₃-C₁₈ alkyl radical is an alkyl radical from the group made up of hexyl, octyl, decyl, and dodecyl.

14- Compound according to any of the claims 6 to 13, characterized in that A is the radical from the dimer diol when it looses the two OH groups.

15- Compound according to claim 14, characterized in that said alkyl radical C₃-C₁₈ is an alkyl radical from the group made up of propyl, butyl, pentyl, hexyl, octyl and decyl, preferably the group made up of butyl, hexyl and octyl.

16- Compound according to any of the claims 2 to 15, characterized in that it comprises a colouring agent, where preferably said colouring agent is linked to said compound by means of an —OH group of the colouring agent and an NCO group of the compound.

17- Compound according to claim 16, characterized in that said compound contains said colouring agent in a proportion less than or equivalent to 0.5% by weight, preferably less than or equivalent to 0.4% by weight with respect to the total weight of the compound.

18- Composition comprising at least a first compound having general formula (I) according to any of the claims 1 to 17, where R, R′, X, X′, A and n have the general meaning indicated above, and a nonpolar solvent, preferably from the group made up of benzene mono- or polysubstituted with C₁-C₄ alkyl radicals, turpentine, and mixtures of the former, where the benzene mono- or polysubstituted with Cₗ-C₄ alkyl radicals is preferably toluene, xylene or ethylbenzene.

19- Composition according to claim 18, characterized in that it comprises at least one second compound having general formula (I), where said second compound has a different molecular weight to said first compound.

20- Composition according to one of the claim 18 or 19, characterized in that it has a solvent content less than or equivalent to 30% by weight with respect to the total weight of the compound having general formula (I), and preferably less than or equivalent to 20% by weight with respect to the total weight of the compound having general formula (I).

21- Composition according to claim 18, characterized in that R and R′ are the radical from the dimer diol when it looses an OH group, X is isophorone, n is 0, and as solvent it has between 11% and 15% by weight of toluene and between 5% and 9% by weight of turpentine, preferably 13% by weight of toluene and 7% by weight of turpentine.

22 - Composition according to claim 18, characterized in that R and R′ are octyl, X is isophorone, n is 0, and as solvent it has between 4% and 8% by weight of toluene and between 7% and 11% by weight of a mixture of benzenes mono- or polysubstituted with C₁-C₄ alkyl radicals, preferably 6% by weight of toluene and 9% by weight of a mixture of benzenes mono- or polysubstituted with C₁-C₄ alkyl radicals.

23- Composition according to claim 19, characterized in that in said first compound R and R′ are the radical from the dimer diol when it looses an OH group, X is isophorone, n is 0, and in said second compound R and R′ are octyl, X is isophorone and n is 0, where the proportion between said first compound and said second compound is between 40:60 by weight and 60:40 by weight, and in that it has between 20% and 35% by weight of said solvent, preferably between 25% and 30% by weight of said solvent.

24- Composition according to claim 18, characterized in that R and R′ are octyl, X is isophorone, n is 0, as solvent it has between 8% and 12% by weight of toluene and between 3% and 7% by weight of a mixture of benzenes mono- or polysubstituted with C₁-C₄ alkyl radicals, preferably 10% by weight of toluene and 5% by weight of a mixture of benzenes mono- or polysubstituted with C₁-C₄ alkyl radicals, and, in addition to the solvent, it has a content between 8% and 12% by weight of octanol.

25- Composition according to claim 18, characterized in that R and R′ are octyl, X is isophorone, n is 0, as solvent it has between 0% and 5% by weight of toluene, and, in addition to the solvent, it has a content between 15% and 25% by weight of octanol.

26- Composition according to claim 18, characterized in that R and R′ are hexyl, X is isophorone, n is 0, and, in addition to the solvent, it has a content between 8% and 12% by weight of hexanol.

27- Composition according to any of the claims 18 to 26, characterized in that it comprises a colouring agent, where preferably it contains said colouring agent in a proportion less than or equivalent to 0.3% by weight, and very preferably less than or equivalent to 0.15% by weight with respect to the total weight of the compound having general formula (I).

28- Method for producing a watermark on a laminar cellulosic material characterized in that it comprises a stage of applying a compound having general formula (I) according to any of the claims 1 to 17, where R, R′, X, X′, A and n have the general meaning indicated above, onto said laminar cellulosic material using the offset system.

29- Method for producing a watermark on a laminar cellulosic material characterized in that it comprises a stage of applying a composition according to any of the claims 18 to 27 onto said laminar cellulosic material using the offset system.

30- Use of a compound having general formula (I) according to any of the claims 1 to 17, where R, R′, X, X′, A and n have the general meaning indicated at the beginning, to produce a watermark on a laminar cellulosic material.

31- Use of a composition according to any of the claims 18 to 27, for producing a watermark on a laminar cellulosic material.

32- Method for producing a watermark on a laminar textile material characterized in that it comprises a stage of applying a compound having general formula (I) according to any of the claims 1 to 17, where R, R′, X, X′, A and n have the general meaning indicated above, onto said laminar textile material using the offset system.

33- Method for producing a watermark on a laminar textile material characterized in that it comprises a stage of applying a composition according to any of the claims 18 to 27 onto said laminar textile material using the offset system.

34- Use of a compound having general formula (I) according to any of the claims 1 to 17, where R, R′, X, X′, A and n have the general meaning indicated above, for producing a watermark on a laminar textile material.

35- Use of a composition according to any of the claims 18 to 27, to produce a watermark on a laminar textile material.
